(19) United States
(12) Patent Application Publication
(10) Pub. No.: US 2021/0353862 A1
(43) Pub. Date: Nov. 18, 2021

(54) MODULAR SYRINGE HOLDER AND SYRINGE ASSEMBLY METHOD

(71) Applicant: Ypsomed AG, Burgdorf (CH)

(72) Inventors: Christian Schrul, Burgdorf (CH); Markus Tschirren, Burgdorf (CH)

(21) Appl. No.: 17/388,507

(22) Filed: Jul. 29, 2021

Related U.S. Application Data
(63) Continuation of application No. PCT/EP2020/052127, filed on Jan. 29, 2020.

(30) Foreign Application Priority Data
Feb. 15, 2019 (EP) 19157362.5

(51) Int. Cl. A61M 5/24; A61M 5/20; A61M 5/32

(52) U.S. Cl. CPC A61M 5/24; A61M 5/2033; A61M 5/3202; A61M 2005/2492; A61M 2005/2407; A61M 2005/2477; A61M 2005/2013

(57) ABSTRACT

Modular syringe holders receive small-volume product containers for use in injection devices configured for nominally large-volume product containers, and include an adapter and an adapter holder configured for insertion in such injection devices. The adapter includes a hollow-cylindrical, rigid adapter body and a radially deflectable support element for a receiving a syringe shoulder of a pre-filled syringe. During receipt of the pre-filled syringe, the support element undergoes a radial deflection by a needle protection cap of the pre-filled syringe. The adapter holder is configured to receive the adapter, and a holding portion of the adapter holder blocks the support element against radial deflection in a holding position in an engagement with the syringe shoulder. With the adapter and pre-filled syringe received in the adapter holder, a flexible shoulder-support element diverts axial forces of the shoulder of the pre-filled syringe and is secured by the adapter holder against lateral deflection.

### CROSS-REFERENCE TO RELATED APPLICATIONS

This application claims priority to International Patent Application No. PCT/EP2020/052127, filed Jan. 29, 2020, entitled “MODULAR SYRINGE HOLDER AND SYRINGE ASSEMBLY METHOD,” which in turn claims priority to European Patent Application No. 19157362.5, filed Feb. 15, 2019, entitled “MODULAR SYRINGE HOLDER AND SYRINGE ASSEMBLY METHOD”, each of which is incorporated by reference herein, in the entirety and for all purposes.

### TECHNICAL FIELD

Implementations relate to the field of medical injection devices for the administration of liquid substances, including drugs or medicinal substances such as insulin and hormone preparations, and more specifically relate to the use of small-volume product containers in injection devices for nominally large-volume product containers.

### BACKGROUND

Injection devices or injection appliances for the simplified administration of a substance include inter alia so-called autoinjectors, which comprise an energy storage or drive element, with which the discharge can be carried out automatically, that is to say without a force to be applied or exerted from the outside by a user. The energy storage or drive element stores the energy necessary for an automatic substance release in a mechanical form. Such an energy storage or drive element can be a spring, which, in a tensed state, is incorporated in the injection device and releases energy due to relaxation. The energy release occurs to a plunger rod or a pressure element, which inserts or drives a plunger in a product container. The energy storage or drive element can also be provided in order to automate the process of insertion of an injection needle. Alternatively, for this purpose, an additional separate drive element can be provided, or the insertion process occurs manually, thus exclusively by a user, without using energy stored in the injection device for this purpose.

The injection device can comprise a product container holder for receiving a product container, where, in the product container holder, the product container can be held in a manner such that it is fixed radially, axially, and preferably also rotationally. The product container holder can be connected in an axially and rotationally fixed manner to the housing of the injection device, or it can be movable relative to the housing during the insertion and/or needle retraction process. The product container can be a carpule for repeated detachable connection with disposable injection needles or a disposable pre-filled syringe with an injection needle which is nondetachably connected therewith. The product container has a hollow-cylindrical product container portion, which shiftably supports a plunger or stopper. The plunger can form a sealing gap with the inner periphery of the product container portion and be shifted by means of the plunger rod in a distal direction in order to discharge a product from the product container via the injection needle.

The injection device can comprise a needle protection sleeve, which protrudes distally over the distal end of the injection needle after the injection has occurred, or which is shifted relative to the housing with relaxation of a needle protection sleeve spring into this position, in order to prevent accidental access to the injection needle and thereby reduce the risk of injury. In an autoinjector, the needle protection sleeve can also be used as a trigger element for triggering the product discharge, where, for this purpose, the needle protection sleeve may be shifted relative to the housing in the proximal direction. Alternatively, triggering of the autoinjector may be through actuating a trigger button of the autoinjector, where the needle protection sleeve 40 may be used at least as sight protection before the use of the autoinjector.

The patent application publication WO2016/205963 A1 describes an autoinjector as an example, comprising a housing with a longitudinal axis, release device, and a product container firmly arranged in the housing. The autoinjector moreover comprises a needle protection sleeve which can be shifted in a longitudinal direction between a proximal and a distal position and which is coupled to a needle protection sleeve spring as separate drive element. A first feedback device with a first support element accelerated by the discharge spring signals the start of the substance release. The second feedback device with a second support element accelerated by the needle protection sleeve spring toward the abutment is used for generating an acoustic signal after the release of a certain quantity of substance. A spiral or drive spring in which energy for the automatic discharge of product can be stored is coupled to the trigger device, wherein a first end of the spiral spring is connected to the housing, and a second end of the spiral spring is connected in a rotationally fixed manner to a rotation element, arranged coaxially to the longitudinal axis, in the form of a threaded rod. The threaded rod engages, via a threading, into a non-rotating advance element in the housing, in the form of a sleeve-shaped plunger rod, which, during a shifting in the distal direction, entrains the stopper of the product container at an at least approximately constant discharge speed. The autoinjector may be configured for pre-filled syringes comprising a product container with a predetermined size and a nominal filling volume of 2.25 mL, where the spiral spring may also suitable for a product to be discharged with a high viscosity of at least 5, or at least 15 cP (0.015 kgm⁻¹s⁻¹). The pre-filled syringe moreover may include a needle which, before use, may be surrounded by an elastic needle guard element and a firm needle protection cap or Rigid Needle Shield (RNS) to ensure the sterility and intactness.

Patent application publication EP 2968063 A1 describes a product container holder or drug container support for a first product or drug container with a first predetermined size, which can contain more than one first volume of a drug. The container support comprises a body and an adapter connected to the body, wherein the adapter is configured to support a second drug container with a second predetermined size, which can contain no more than the first volume of the drug. The second drug container is secured in an axially and rotationally fixed manner to the adapter, and the adapter in turn is held in an axially and rotationally fixed manner in the container support. For this purpose, the adapter comprises an arm with a first and a second support surface, which engages with a proximal flange of the second drug container. The adapter has a peripheral, non-flexible distal rib or support surface, against which a distal end or a shoulder of the second drug container rests and which receives an axial force acting on the drug container via the stopper.

The Patent application publication WO 2012/164389 A2 describes, in FIGS. 13 to 15, a similar adapter with flexible fingers, which are arranged distally on a sleeve and which comprise, on their end, a proximally directed support surface for a product container shoulder. In the assembly of a pre-filled syringe, its needle protection cap is pressed through an opening formed by the fingers, wherein the fingers are deflected radially outward. Subsequently, the securing ring is shifted forward, and, as a result, the fingers are deflected again inward and the support surfaces are blocked in a holding position in engagement with the product container shoulder. Thereby, in the blocked state, the opening formed by the inner edges of the support surfaces has a diameter which is smaller than an outer diameter of the needle protection cap. The support surfaces then are arranged at least partially between the needle protection cap and the product container shoulder and also enable a receiving of axial forces of the product container shoulder, if their outer diameter is only insignificantly greater than or not greater than the diameter of the needle protection cap.

In this context, the term “product,” “drug,” or “medical substance” comprises any flowable medical formulation which is suitable for the controlled administration by means of a cannula or a hollow needle, for example, of a liquid, a solution, a gel or a fine suspension containing one or more medical active substances. A drug can thus be a composition with a single active substance or a premixed or co-formulated composition with a plurality of active substances from a single container. In particular, the term covers medicines such as peptides (for example, insulins, insulin-containing drugs, GLP-1-containing as well as derived or analogous preparations), proteins and hormones, biologically prepared or active substances, active substances based on hormones or genes, nutrition formulations, enzymes and additional substances in solid (suspended) or liquid form. The term moreover also covers polysaccharides, vaccines, DNA or RNA (including mRNA) or oligonucleotides, antibodies or parts of antibodies as well as suitable base, adjuvant, and carrier substances.

The term “distal” refers to the front, insertion-side end of the administration device, or to the side or direction directed toward the tip of the injection needle. On the other hand, the term “proximal” refers to a side or direction directed toward the rear end of the administration device, which is opposite the insertion-side end.

In the present disclosure, the terms “injection system” or “injector” is understood to refer to a device in which, after a controlled quantity of the medical substance has been discharged, the injection needle is removed from the tissue. Thus, in an injection system or in an injector, in contrast to an infusion system, the injection needle does not remain in the tissue for a longer time period of several hours.

### SUMMARY

Implementations provide product containers with a small filling volume in an injection device for typical use with product containers with a large filling volume by providing modular syringe holders, and methods for assembling injection devices including such modular syringe holders.

According to implementations, a modular syringe holder may include an adapter and an adapter holder for holding a ready-to-use or pre-filled syringe in a syringe unit of an injection device with a housing, which housing may be gripped by a user and defines a longitudinal axis. The adapter holder may differ from the housing and the needle protection sleeve of the injection device and may be introduced in a separate assembly step into the housing, where the adapter holder may be held in an axially and rotationally fixed manner. The adapter may include a hollow-cylindrical, rigid adapter body, a support element coupled to the adapter body which may be at least radially deflectable, and the support element may be configured for receipt of or contact with a syringe shoulder of the pre-filled syringe, where the syringe shoulder may be formed distally with respect to a hollow-cylindrical product container portion by a radial narrowing of the syringe body. The adapter may be configured or prepared for introducing the pre-filled syringe in the direction of the longitudinal axis with a deflection of the support element of the adapter by a needle protection cap of the pre-filled syringe. The adapter holder may be configured or prepared for receiving the adapter, such that the support element may be blocked by a rigid holding portion of the adapter holder and not, for example, by the housing of the injection device, against the radial deflection or a shearing in a holding position of the support element of the adapter in which the support element may be in an engagement with the syringe shoulder.

A flexible shoulder extension, such as a support element, may be configured for diverting axial forces of a product container, and after the reception of the adapter and the product container in the adapter holder, may be secured by the adapter holder itself against lateral deflection, and may not require a manual shifting of a separate securing ring in the distal direction for this purpose. A corresponding assembly method may involve the steps of:
inserting the adapter holder into the syringe unit,
introducing the pre-filled syringe into the adapter, and
introducing or shifting the adapter holding the pre-filled syringe into the injector unit.

In some implementations, the adapter of the modular syringe holder may include a flexible finger with an at least approximately constant cross section, which may be fastened on the adapter body and which may support, on its distal end, the support element configured for receiving distal axial forces of the syringe shoulder of a pre-filled syringe assembled therein. In the direction of rotation about the longitudinal axis L, e.g., in a direction transverse to the longitudinal axis, the extension of the support element may be greater than the corresponding extension or width of the flexible finger, and the support element may protrude at least in one direction of rotation and may protrude in both directions of rotation away from the flexible finger. The support element may include a section or a segment of a circular ring with an average diameter corresponding to a diameter of the syringe shoulder; and an associated arc length may exceed the width of the finger. Due to this combination, a relatively enlarged support surface of the support element and a pre-defined mobility or flexibility of the flexible finger may be provided at the same time.

In some implementations, the adapter and the adapter holder may be configured such that, in the assembled state of the syringe holder in the injection device, the support element may be directly in contact, in the axial direction, with a proximally oriented surface of the housing. Axial forces may thus be transmitted from the syringe shoulder to the support element and from a distally directed front side of the support element directly to the housing, and may not be transmitted first onto the adapter holder. This may facilitate avoiding holding the syringe in a proximal position, where for instance, in the case of a predetermined needle length, a desired insertion depth may not be reached. For this purpose, the adapter holder may include, on the distal end, a minimum inner diameter, which may be greater than an outer diameter of the adapter or of the support element of the adapter.

In some implementations, the adapter holder and the housing of the injection device may include securing elements such as catches or flexible arms, which may engage in recesses, and may fix the introduced adapter holder in the housing in an axially and rotationally fixed manner. In addition, the adapter and the adapter holder may be configured such that, in the assembled state of the syringe holder in the injection device, the pre-filled syringe may rotate about the longitudinal axis. The pre-filled syringe such as a finger flange of the syringe may thus not be prevented from rotating by the housing or by the adapter holder assembled in a rotationally fixed manner in the housing. The adapter itself may be received in a rotationally fixed manner in the adapter holder, and a proximal end of the adapter may be sufficiently widened and/or spaced from the finger flange and may enable a rotation of the pre-filled syringe. A syringe freely rotatable in the injection device may avoid transmitting a rotation or rational forces of the injection device to the injection site and as a result may cause less pain in the patient for instance during injection and/or needle insertion. A lateral widening or broadening of the adapter or adapter holder, which may be provided for enabling free rotation of the syringe, may additionally be used as a front-side or proximal end support in the housing.

In some implementations, the adapter of the modular syringe holder may include two flexible fingers, which may be coupled or fastened to the adapter body. On their distal ends, each of the flexible fingers may support a support element for receiving distal axial forces of the syringe shoulder of a pre-filled syringe assembled therein. Alternatively, the support element may include a flexible collar directed radially inward, which may be interrupted by at least one slot or gap in the radial direction and which may be widened temporarily by for example an insertion of a needle protection cap of the pre-filled syringe. In some implementations, a diameter of a distal opening of the adapter, which may be formed by the support element(s), may be smaller than the maximum outer diameter of the needle protection cap of the pre-filled syringe. In addition, an axial length of the support element at each support point of the syringe shoulder may be smaller than an axial distance between a proximal end of the needle protection cap and the support point of the syringe shoulder. As a result, the syringe may contact and be distally held by the support element of the adapter, the needle protection cap may not be loaded by the support element, and an accidental movement or shifting of the needle protection cap by the adapter may be avoided, which may otherwise jeopardize the sterility of the needle.

According to implementations, a method for assembling a pre-filled syringe in a syringe unit of an injection device housing defining a longitudinal axis, the housing for example configured to be gripped by a user, may include the following steps:
inserting a syringe holder with a rigid holder sleeve and a flexibly coupled support element into an injection unit of the injection device in a first axial position, in which the support element can be deflected outward,
blocking the syringe holder against a movement in the distal direction,
introducing or shifting a pre-filled syringe into the syringe holder in the distal direction which may include a radial deflection of the support element by a needle protection cap of the pre-filled syringe,
releasing or unblocking a movement of the syringe holder in the distal direction, and
moving or advancing the syringe holder containing the ready-to use syringe into a second axial position in which the support element may be blocked by a holding portion of the housing against radial deflecting or shearing in a holding position in engagement with the syringe shoulder.

For the introduction of the pre-filled syringe into the syringe holder, the syringe holder may be out of engagement with the holding portion of the housing. When the pre-filled syringe is inserted completely into the syringe holder and the support element is engaged in a gap between the syringe shoulder and the needle protection cap, the syringe holder may be brought in engagement with the holding portion of the housing. In this engagement position, the support element may be prevented from moving transversely or deflecting radially relative to the longitudinal axis out of the engagement with the syringe holder. A support element configured as a flexible shoulder for diverting axial forces of a product container may thus also be secured here against lateral deflection after the reception of the syringe holder and of the product container in the housing by the housing itself, for instance without requiring a manual shifting of a separate securing ring in the distal direction for this purpose.

According to implementations of the present disclosure, in the assembly method, the blocking of the syringe holder may occur via use of an assembly tool, which may engage on a distally oriented assembly supporting element of the syringe holder. For example, the assembly tool may be introduced laterally through an inspection window in the housing into a region of the syringe holder, and, after shifting of the syringe holder until the support element is in abutment with the assembly tool, the assembly tool may be removed, for instance to unblock movement of the syringe holder after the pre-filled syringe is introduced into the syringe holder and for completion of the assembly method.

### BRIEF DESCRIPTION OF THE DRAWINGS

In connection with the appended figures, implementations of the present disclosure are described herein. They are intended to show basic possibilities of the disclosed implementations and should in no way be interpreted to be limiting.

FIG. 1 shows an exploded isometric view of an autoinjector according to the present disclosure;

FIG. 2 shows a cross-sectional view of the autoinjector of FIG. 1 in the delivery state;

FIG. 3 shows an isometric view of a one-piece syringe holder according to the present disclosure;

FIGS. 4a-4c show an assembly process of a pre-filled syringe in the syringe holder of FIG. 3;

FIG. 5a shows an isometric view of an adapter of a two-piece syringe holder;

FIG. 5b shows an isometric view of an adapter holder of the two-piece syringe holder;

FIG. 6 shows a cross-sectional view of the two-piece syringe holder of FIGS. 5a and 5b in a delivery state;

FIG. 7a shows an exploded isometric view of the housing and the end cap of the autoinjector of FIG. 1;

FIG. 7b shows a second isometric view of the end cap of the autoinjector of FIG. 1;

FIG. 8 shows an exploded isometric view of the components of a spring pack of the autoinjector of FIG. 1;

FIGS. 9a-9c show isometric views of the spring pack of FIG. 8;

FIG. 10 shows an isometric view of the spring pack of FIG. 8 and of a preassembled drive unit according to implementations of the present disclosure; and

FIGS. 11a-11b show isometric views of a second spring pack according to implementations of the present disclosure.

### DETAILED DESCRIPTION

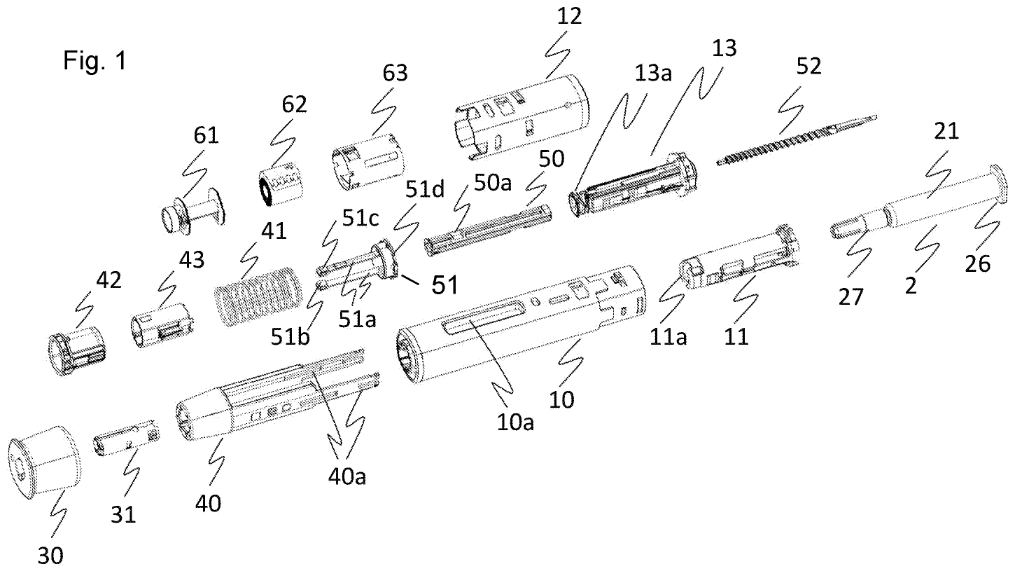

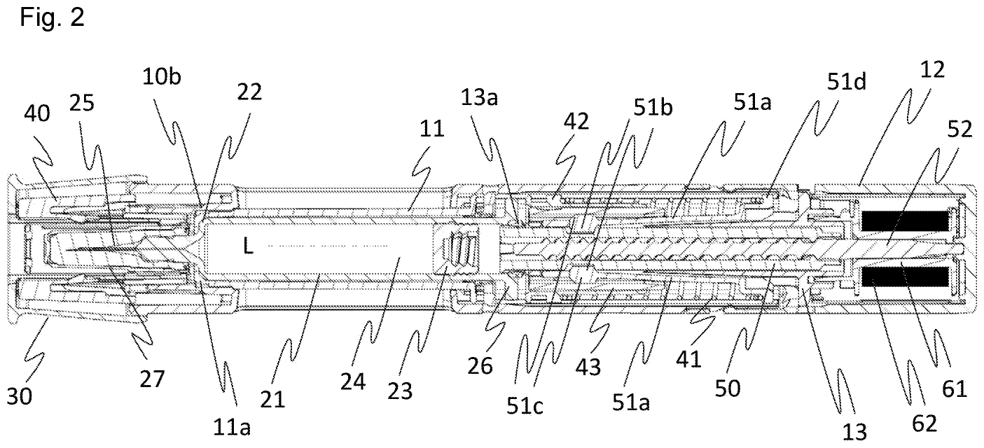

FIG. 1 shows an exploded isometric view and FIG. 2 shows a longitudinal view of an injection device (e.g., an autoinjector) of the present disclosure with a sleeve-shaped cylindrical housing 10 with a longitudinal axis L, on which a syringe holder 11, an end cap 12, and a mechanism holder 13 may be firmly arranged in a delivery state of the autoinjector. For this purpose, the syringe holder 11, the end cap 12 and the mechanism holder 13 may be engaged, snapped or otherwise assembled in a rotationally and axially fixed manner to the housing 10.

In the syringe holder 11, a pre-filled syringe 2 may be received and held therein. The pre-filled syringe 2 may include a cylindrical syringe body 21 that may be configured as a product container, which may define a product receiving space 24 between a syringe shoulder 22 and a piston or stopper 23, where the stopper may be shifted along the longitudinal axis L (FIG. 2). The syringe shoulder 22 may include a tapering syringe section with a cross-section that may be smaller in comparison to the syringe body 21. On a distal end of the syringe body 21, a hollow injection needle 25 may be connected, e.g., non-releasably connected, to the syringe body 21 and may be connected or extend from the syringe shoulder 22; and on a proximal end of the pre-filled syringe 2, a finger flange 26 may be attached or be formed, which may protrude radially outward beyond an outer circumference of the syringe body 21. In the delivery state, the product receiving portion 24 may contain a product to be delivered, which may be expelled from the product receiving portion 24 through the injection needle 25 by shifting the piston 23 in a delivery or expelling direction from a piston start position into a piston end position.

The injection needle 25 of the pre-filled syringe 2 may be covered by a needle protection cap 27, which may be configured as a so-called Rigid Needle Shield (RNS) and may include a flexible or rubber-elastic needle guard element and a sleeve made of hard plastic. The needle protection cap 27 may protect the injection needle 25 from unintentional detachment or other mechanical actions and from soiling and thus may maintain the sterility of the injection needle and the product contained in the pre-filled syringe 2. Between the syringe shoulder 22 and the proximal end of the sleeve of the needle protection cap 27, at least a portion of which may be made of hard plastic, a gap may be formed. On the distal end of the autoinjector, in the delivery state thereof, a pull cap 30 may be arranged, which, before the use of the autoinjector, may be axially pulled and/or twisted off and completely removed. The pull cap 30 may include snap hooks or a separate protection cap remover 31 that may at least be axially held in the pull cap 30, and the snap hooks or protection cap remover 31 may be arranged in the gap and removal of the pull cap 30 may result in release of the needle protection cap 27 from the pre-filled syringe 2.

The injection needle 25 may be surrounded by a needle protection sleeve 40 mounted in an axially shiftable manner relative to the housing 10 and insertable or slidable into the housing 10. In the starting position of the needle protection sleeve 40, the distal end of the needle protection sleeve 40 may protrude distally over the needle tip of the injection needle 25, so that access to the needle tip may be initially prevented. The needle protection sleeve 40, at a distal or front side, may include an opening through which the injection needle 25 may protrude, and during a relative movement of the needle protection sleeve 40 and the injection needle 25, the injection needle 25 may enter through the opening and into an injection site. The needle protection sleeve 40 may also be used as a trigger element for triggering the product discharge, where, for this purpose, the needle protection sleeve 40, which may be biased by a needle protection sleeve spring 41, may be shifted relative to the housing 10 in the proximal direction. For instance, the needle protection sleeve 40 may include two sleeve arms 40a, which may be arranged offset or rotated by 90° about the longitudinal axis L with respect to two inspections windows 10a or two recesses of the housing 10, which may be referred to as inspection windows. After an injection has occurred, the needle protection sleeve 40 may be shifted relative to the housing 10 from the actuated position along the longitudinal axis L in the distal direction into a needle protection position and may be blocked from being pushed back in the proximal direction.

The autoinjector may include a switching module with a switching sleeve 42 and a blocking sleeve 43 surrounded by the switching sleeve 42. The switching sleeve 42 may be connected to a proximal end of the sleeve arms 40a of the needle protection sleeve 40 and to a distal end of the needle protection sleeve spring 41. The needle protection sleeve spring 41 may be formed as a spring made of metal and may serve as a compression spring and may be configured as a coil spring.

The autoinjector may include a drive with an axially shiftable plunger rod 50 that may be configured as an advancing element for moving the piston 23 in the discharge direction. The autoinjector may include a holding element 51 with two flexible holding arms 51a, where, on a distal end of each holding arm 51a, a first engagement element 51b and a second engagement element 51c may be arranged. The first engagement element 51b may extend radially toward the longitudinal axis L, and the second engagement element 51c may extend radially away from the longitudinal axis L. In the delivery state of the device, the first engagement element 51b may be held in an engagement with a recess 50a of the plunger rod 50 by the inner periphery of the blocking sleeve 43, which may be in contact with the second engagement element 51c, where a movement of the plunger rod 50 relative to the holding element 51 in the discharge direction may be prevented. The needle protection sleeve spring 41 may be supported by its proximal end on the holding element 51, for instance on a projection 51d of the holding element 51, which may engage in an axially shiftable and rotationally fixed manner in the housing 10.

The drive may additionally include a spring pack 6 (FIG. 9b), which may be configured as a preloaded energy store, and may be assembled in the autoinjector for delivering a driving force for causing an injection and/or product discharge. The spring pack 6 may include a spring shaft 61, a spiral or drive spring 62, which, in the delivery state, may store at least the amount of energy needed for a complete discharge of the product in the product receiving portion 24 of the pre-filled syringe 2, and a spring sleeve 63. The drive may additionally include a threaded rod 52 (FIG. 2), which may be configured as a rotational element, and which may be coupled to an inner end of the spring coil 62 and may be in an engagement with an inner threading of the plunger rod 50 such that a rotation of the threaded rod 52 may result in transmitting the energy of the spring sleeve 63 to the plunger rod 50, and the plunger rod 50 may thereby be moved in the distal direction for product discharge. The spiral spring 62 may be wound from a strip-shaped material, such as a spring steel.

The pre-filled syringe 2 may be received in the syringe holder 11 and may be secured at least against a movement along the longitudinal axis L in the distal direction relative to the syringe holder 11. For instance, the syringe holder 11 may include at least one inwardly protruding and proximally directed axial support element 11a on which the spring shoulder 22 may be supported against movement in the distal direction. In order to prevent a proximal movement of the pre-filled syringe 2, the pre-filled syringe 2 may be pressed by a holding spring portion 13a of a mechanism holder 13, which may engage on the finger flange 26 of the pre-filled syringe 2, into an engagement with the support element 11a. In some implementations, the holding spring portion 13a may account for longitudinal differences of the syringe body 21, which may be generated due to manufacturing tolerances. Between the finger flange 26 and the proximal end of the syringe holder 11, a gap may be formed. The housing 10 may include an annular peripheral holding portion 10b (FIG. 2), which may surround the distal end of the assembled syringe holder 11 in an annular manner and which may secure the syringe holder 11 in the region of the axial support element 11a against radial deflections.

The pre-filled syringe 2 represented in FIG. 1 and FIG. 2 may be configured with a product container with a predetermined size and a nominal filling volume of 2.25 mL, where the syringe body 21 may include an outer diameter which may be larger than the needle protection cap 27, and the support elements 11a of the syringe holder 11 may have a rigid configuration, e.g., a non-flexible configuration. As disclosed further herein, two implementations are provided for use with autoinjectors configured for large-volume pre-filled syringes, in which smaller-volume pre-filled syringes with a product container having a smaller size and a smaller outer diameter may be used with minimal adjustments.

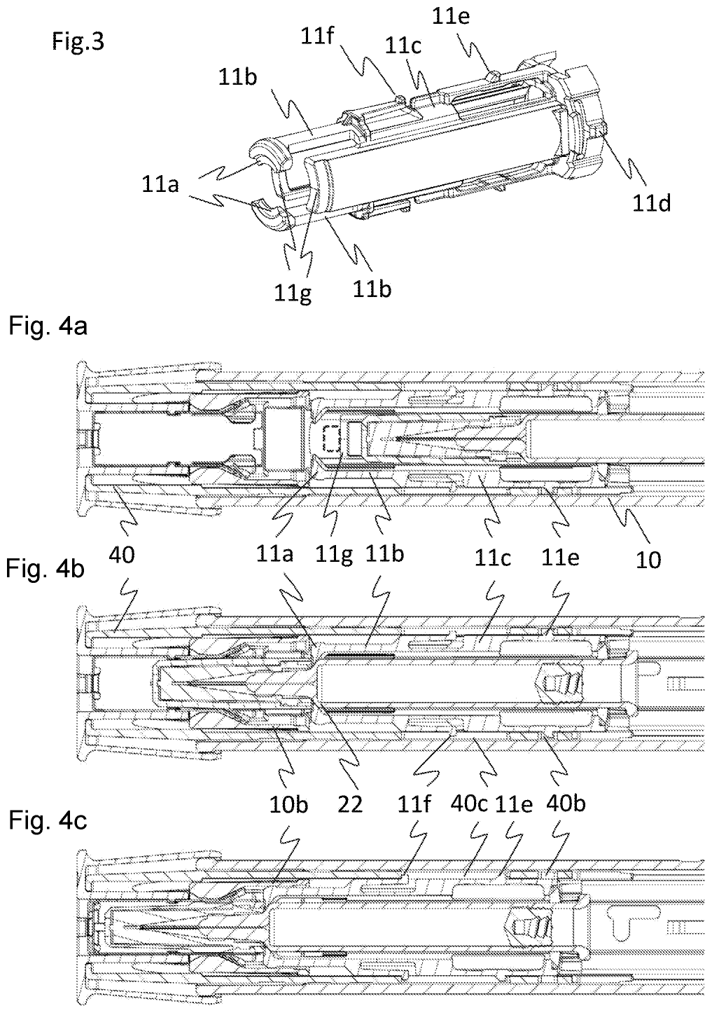

FIG. 3 shows a first implementation of the present disclosure in which a one-piece syringe holder 11 is provided. The one-piece syringe holder 11 may be suitable for holding a pre-filled syringe 2 with a small product container and therefore small product container volume in an autoinjector configured for nominally larger product container volumes. The one-piece syringe holder 11 may include two elastic fingers 11b which, on their proximal ends, may be attached to a holder sleeve 11c of the syringe holder and which, on their distal ends, may each include an axial support element 11a for a syringe shoulder 22. Moreover, on the holder sleeve 11c, two first snap elements 11d, which may be arranged on opposite sides of the holder sleeve 11c, may be attached for engagement in the housing 10 as well as in each case two second and third snap elements 11e, 11f, which may be arranged on opposite sides of the holder sleeve 11c, for engagement in recesses of the needle protection sleeve 40. Features of the support elements 11a and functions of the snap elements 11e, 11f, which may apply to multiple variants are summarized after the description of the different embodiments.

FIGS. 4a-4c show three steps of an assembly of the one-piece syringe holder 11 of FIG. 3 into the housing 10, according to implementations of the present disclosure. First, the one-piece syringe holder 11 may be introduced axially into the housing 10 and held in a first axial position (FIG. 4a). In this first axial position, the distal ends of the fingers 11b of the one-piece syringe holder 11 with its support elements 11a may be deflected radially outward and may form an opening sufficient for passage of the needle protection cap 27 therethrough. For instance, the fingers 11b may not be prevented from a radial outward movement by a holding portion 10b of the housing 10. The one-piece syringe holder 11 may be temporarily held in the first axial position by a readily detachable snap connection between the one-piece syringe holder 11 and the housing 10, and for instance may include the two second radially outward pointing snap elements 11e of the one-piece syringe holder 11 and the first recesses 40b of the needle protection sleeve 40 which may be configured complementary to the snap elements 11e. For instance, the snap elements 11e may include distal holding surfaces with a beveled form and may be insufficient for receiving the forces acting on the one-piece syringe holder 11 when the pre-filled syringe 2 is pressed in, such that, for this step, an assembly tool may be introduced from outside into the housing 10 and engaged with assembly supporting elements 11g of the syringe holder 11. The assembly supporting elements 11g may be formed by suitable, distally directed, rigid surfaces, edges, projections, cams or other protrusions on the holder sleeve 11c. For this purpose, in the implementation of FIG. 3, two edges may be provided on the distal end of the holder sleeve 11c, rotated about the longitudinal axis L by 90° between the fingers 11b or with respect to the fingers 11b. These edges may be offset sufficiently far with respect to the support elements 11a in the proximal direction, so that in each case an opening forms in the first axial position between the edges and the distal frame of the inspection window 10a. Through these openings, a two-piece assembly tool may be introduced from both sides of the exterior and into the housing 10 at a right angle with respect to the longitudinal axis L, e.g., in a straight line, up to an engagement with the assembly supporting elements 11g. The axial position of the assembly tool is illustrated diagrammatically by the dot-dash lines in FIG. 4a; and in the process of insertion, the assembly tool may not protrude radially into an inner space of the one-piece syringe holder 11.

As second step, the pre-filled syringe 2 may be axially inserted into the one-piece syringe holder 11 (FIG. 4b), where the support elements 11a may first be pushed radially outwards and subsequently engage behind the needle protection cap 27 in the gap formed between the needle protection cap 27 and the syringe holder 22. Subsequently, the assembly tool may be removed from engagement with the one-piece syringe holder 11. Finally, the one-piece syringe holder 11 and the pre-filled syringe 2 together may be distally shifted into a second axial position (FIG. 4c). In this position, the flexible fingers 11b may be held with the support elements 11a by the holding portion 10b of the housing 10 with an inner diameter which may be smaller in comparison to the first position, and the flexible fingers 11b and support elements 11a may be blocked against radial deflection or shearing by the holding portion 10b of the housing 10. The one-piece syringe holder 11 may be snapped into the second axial position by the two first snap elements 11d snapping into recesses of the housing 10, which may be complementary thereto, in a manner so the snap elements 11d cannot be released by further axial movements. By distally shifting the one-piece syringe holder 11 and the pre-filled syringe 2 together into the second axial position, the needle protection cap remover 31 may be pressed in the distal direction, e.g., without play, against at least one abutment on the pull cap 30.

Instead of the two assembly supporting elements 11g, which may be arranged opposite one another on the one-piece syringe holder 11, a single assembly supporting element or more than two assembly supporting elements may also be provided. The process of supporting the one-piece syringe holder 11 may involve configuring one or more assembly supporting elements that are adapted to suitable recesses in the housing 10 and/or to capabilities of the assembly tool, with the goal of enabling a simple or streamlined intermediate assembly step, e.g., involving the first and second assembly steps. In implementations when the assembly tool can be positioned before the introduction of the one-piece syringe holder 11, the syringe holder 11 may be configured without the second snap elements 11e when otherwise used in connection with positioning the one-piece syringe holder 11 in the first axial position (e.g., FIG. 4a).

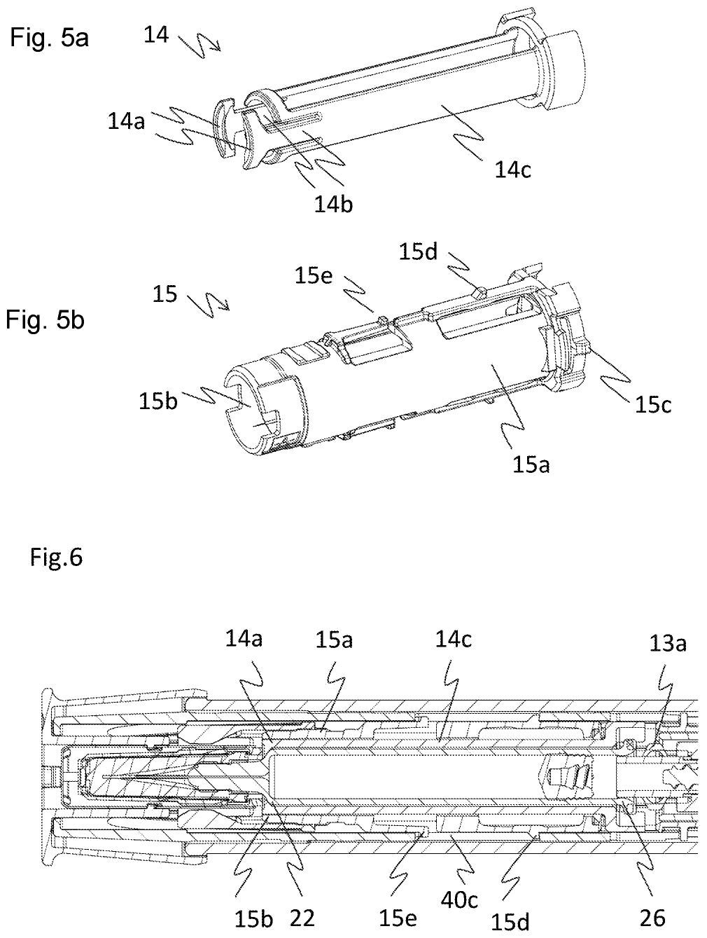

FIGS. 5a, 5b, and 6 show a second implementation of the present disclosure in which a two-piece or modular syringe holder is provided that may include an adapter 14 and an adapter holder 15, which may be suitable for holding a smaller pre-filled syringe in an autoinjector for nominally larger product container volumes. The adapter 14 (FIG. 5a) may include an adapter body 14c with an inner diameter adapted to receive an outer diameter of small-volume pre-filled syringes; and the outer diameter of the adapter body 14c may, for instance, correspond to an inner diameter of a holder sleeve 15a of the adapter holder 15 (FIG. 5b). In the assembled state of the syringe holder (FIG. 6), the adapter body 14c may receive the syringe body 21 of the pre-filled syringe, while the holder sleeve 15a may radially enclose the adapter body 14c and may be held in the housing 10 of the autoinjector. The adapter holder 15 differs from a syringe holder of the injection device for a large-volume pre-filled syringe, and for instance the inner diameter of the adapter holder 15 may be selected to be greater or smaller than an outer diameter of the large-volume pre-filled syringe.

The adapter body 14c of the adapter 14 may include two adapter arms which may be connected on their distal end via webs and on their proximal end via an extension. The arms may define and delimit, in the direction of the longitudinal axis L, two longitudinal recesses in the adapter body 14c, which, in the assembled state, each of the recesses may be aligned with one of the inspection windows 10a of the housing 10 and may thereby avoid obstructing a view into the syringe body 21 of the pre-filled syringe 2. The proximal extension may form an adapter shoulder and an inner space with a diameter, which may be enlarged with respect to the adapter body, for receiving the finger flange 26 of the pre-filled syringe 2. The adapter 14 moreover may include two elastic fingers 14b, which may be attached on their proximal ends to the adapter body 14c and which may each include, on their distal ends, an axial support element 14a for supporting the syringe shoulder 22 of the pre-filled syringe 2.

The holder sleeve 15a of the adapter holder 15 may include an annular peripheral holding portion 15b on its inner side, which may surround the distal end of the assembled adapter 14 in a circumferential manner, and which may secure the support elements 14a against radial deflections. The distal end of the holder sleeve 15a may not include holding elements or narrowings. In this manner, the adapter 14 may rest with its distal ends of the support elements 14a directly against the housing 10 of the autoinjector, and axial forces may be diverted onto the product container of the pre-filled syringe 2 from the syringe shoulder 22 via the support elements 14a to the housing 10. In this position, the finger flange 26 of the pre-filled syringe 2 may not be in contact with the adapter shoulder of the adapter body 14c and the pre-filled syringe 2 may not be prevented from rotating by the adapter extension or by the adapter holder 15 or another housing portion, so that the assembled pre-filled syringe 2 may freely rotate. On the holder sleeve 15a, two first snap elements 15c or securing elements may be provided and may be arranged opposite one another and the snap elements 15c may be used for engagement in the housing 10; and two second and third snap elements 15d, 15e or securing elements, each of which may be arranged opposite one another may be used for engagement in recesses of the needle protection sleeve 40.

For assembly of the pre-filled syringe 2, the adapter holder 15 may be first introduced into the housing 10 of the autoinjector and snapped with two mutually opposite first snap elements 15c into recesses of the housing 10, which may be complementary thereto, in an axially nondetachable and rotationally fixed manner. Subsequently, outside of the injection device, the pre-filled syringe 2 may be introduced axially in the distal direction into the adapter 14. In the process, the needle protection cap 27 may be pressed through an opening formed by the fingers 14b, where the fingers 14b may be deflected radially outward through the sleeve of the needle protection cap. As soon as the proximal end of the needle protection cap has cleared the opening, the fingers may snap radially inward, e.g., into the gap formed between the needle protection cap 27 and the syringe shoulder 22 of the syringe body 21, and position the support elements 14a in a holding position in engagement with the syringe shoulder 22. Finally, the adapter and the syringe may be introduced in a rotatably correct position into the adapter holder. In order to prevent a proximal movement of the pre-filled syringe 2, the pre-filled syringe may be pressed by a holding spring portion 13a of the mechanism holder 13, which may engage on the finger flange 26, into engagement with the support element 14a.

In the two implementations described herein, each support element 11a, 14a may include a portion or a section of a circular ring with an average diameter corresponding to a diameter of the syringe shoulder 22. The support element 11a, 14a may be coupled to a distal end of their respective finger 11b or 14b and may protrude from the finger in a direction transverse to the longitudinal axis. For example, the support element 11a, 14a and/or a central arc length of the section thereof may be greater than a width of the finger (11b, 14b) The support element 11a, 14a may protrude in both directions of rotation from the finger (11b, 14b), and the finger and the support element may together define the shape of an anchor. During the assembly of a pre-filled syringe 2, the needle protection cap 27 of the pre-filled syringe 2 may be pressed through the opening formed by the fingers 11b, 14b, where the fingers 11b, 14b may be deflected radially outward by the sleeve portion of the needle protection cap 27. As soon as the proximal end of the needle protection cap 27 has cleared the opening, the fingers 11b, 14b may snap radially inward and position the support elements 11a, 14a in a holding position in engagement with the syringe shoulder 22. Thereby, the opening formed by the inner edges of the support elements 11a, 14a may be in an engagement with the pre-filled syringe 2 and may have a diameter which is smaller than an outer diameter of the needle protection cap 27. The support surfaces of the support elements 11a, 14a may thus be arranged at least partially in the gap between the needle protection cap 27 and the syringe shoulder 22, and may also make it possible for axial forces of the pre-filled syringe 2 to be received by the support elements 11a, 14a when an outer diameter of the pre-filled syringe 2 is not significantly greater than, or not greater than, the outer diameter of the needle protection cap. By means of the holding portion 10b, 15b of the respective housing 10 or of the adapter holder 15, which may ensure engagement and which may rest with little play against an outer periphery of the fingers 11b, 14b, the fingers may be prevented from moving transversely to the longitudinal axis, and, in the process, may prevent compromising the axial support provided by the support elements 11a, 14a against the syringe shoulder 22.

As an alternative to providing two support elements 11a, 14a on two fingers 11b, 14b, one support element may instead be provided in the form of a radially inward directed collar or a narrowing, which may be temporarily widened by the needle protection cap. The collar may be discontinuous and may include a radial slot to form a C-shaped circular arc section spanning over 180°, which may be elastically spread apart, e.g., perpendicular to the longitudinal axis L, by the needle protection cap 27 and may be pushed away. In addition or alternatively, more than two fingers and/or, instead of anchor-shaped support elements, only support elements having the width of the respective finger may be provided. The spatial volume in which the finger(s) 11b of the first embodiment may be deflected when the pre-filled syringe 2 is introduced, and/or the holding portion 10b, 15b of the respective housing 10 and of the adapter holder 15 may be adjusted in accordance with the number of fingers and the extension of the support elements and/or in terms of shape. Instead of the elastic fingers, flexible non-elastic fingers may instead be provided, for example, with a joint, such as a film joint or flexible hinge joint, in the transition to the holder sleeve 15a or to the adapter body 14c. In this case, the support element 11a, 14a may be guided first by appropriate inclinations on the proximal end of the holding portion 10b of the housing 10 and of the adapter holder 15 during the final movement of the pre-filled syringe 2 into engagement with the syringe shoulder 22.

By the use of a syringe holder 11 according to the first implementation, or of a modular syringe holder 14, 15 according to the second implementation, an injection device configured for nominally large-volume product containers may also be used with small-volume product containers. The transition to a product container with a smaller diameter and a shorter length according to the present disclosure may additionally require at least, or at most, an exchange of a plunger rod and of a mechanism holder. A plunger rod with a smaller diameter may fit into the smaller product container of the present disclosure, and a longer mechanism holder and/or a longer holding spring portion may also be capable of distally pushing with sufficient force a shorter product container in an engagement with the support element 11a, 14a of the present disclosure.

The syringe holder 11 of the first implementation and the adapter holder 15 of the second implementation may be provided for subsequent add-on functions in the assembled state. For this purpose, the second snap elements 11e, 15d of the respective syringe holder 11 and of the adapter holder 15 may engage in a complementary manner with second slot-shaped recesses 40c of the needle protection sleeve 40. In the starting position of the needle protection sleeve 40 (FIGS. 4c and 6) and/or in the needle protection position of the needle protection sleeve 40, the proximal ends of the slot-shaped recesses 40c may rest against proximal stop surfaces of the second snap elements 11e (FIG. 4c), 15d (FIG. 6), where a movement of the needle protection sleeve 40 in the distal direction may be prevented. The third snap elements 11f, 15e of the respective syringe holder 11 and of the adapter holder 15 for instance in the form of cams may be arranged resiliently on the respective holder sleeve 11c, 15a and may also engage in the second recesses 40c, or alternatively in another slot-shaped recess of the needle protection sleeve 40. The third snap elements 11f, 15e, e.g., cams, may be formed so that during an attempt to shift the needle protection sleeve 40 from the starting position into the housing 10 to the actuated position, the snap elements third 11f, 15e may first prevent the shifting of the needle protection sleeve 40. As soon as the force applied for shifting back exceeds a certain threshold value, the third snap elements 11f, 15e may be pushed radially inward out of engagement by the distal ends of the recess 40c, where the needle protection sleeve 40 may be abruptly shifted into the actuated position and the injection needle 25 may be inserted into the puncture site.

The first, second and third snap elements 11d, 11e, 11f and 15c, 15d, 15e may each be provided in pairs on mutually opposite sides of their respective syringe holder 11 and of the adapter holder 15, where in each case, the use of only one element or multiple snap elements distributed over a periphery may also be provided as an alternative. The syringe holder 11 and the adapter holder 15 may be made of a plastic having, in comparison to the housing 10, different material properties, for instance to facilitate the formation of the elastic snap elements 11d, 11e, 11f and 15c, 15d, 15e. In contrast to the housing 10, the syringe holder 11 and the adapter holder 15 may also be optically transparent, and as a result, may facilitate protecting the product container from breakage or shattering without obscuring the visibility of the product through the recesses of the inspection windows 10a in the housing 10.

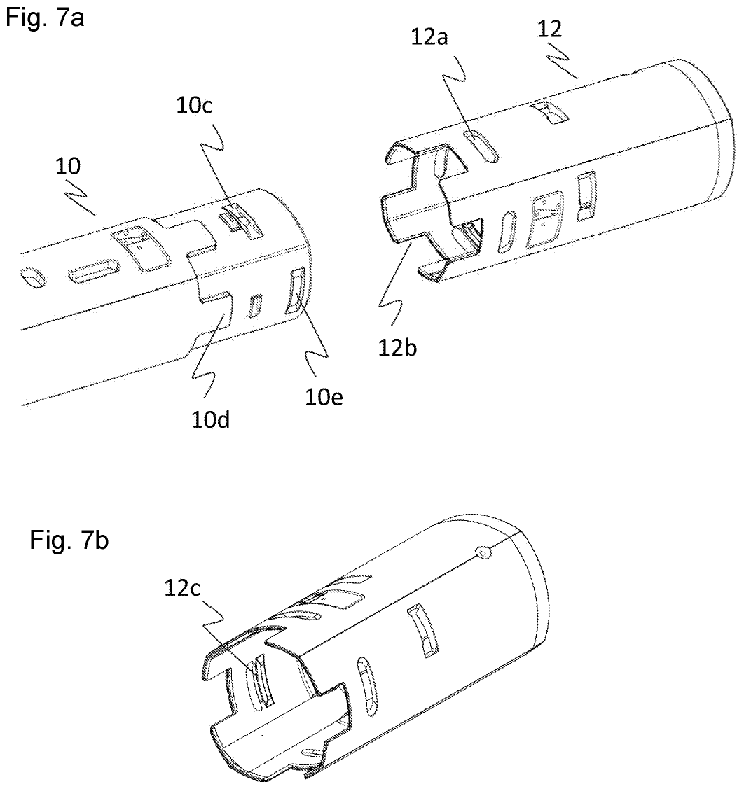

FIG. 7a shows a perspective view of the housing 10 and of the end cap 12 in axially separated position and in a correct rotation position for their final assembly. In the delivery state, the housing 10 and the end cap 12 may be connected to one another with a positive-locking connection in a rotationally and axially fixed manner so that the end cap 12 closes the housing 10. For this purpose, the housing 10 may include snap elements 10c, which may engage in openings or recesses 12a of the end cap 12 (FIG. 7b) and may ensure at least an axial locking. In the direction of the longitudinal axis L, the distal end of the end cap 12 may have a non-rotationally symmetric cap contour 12b, with which may be matched with a complementary housing contour 10d of the housing 10, which may ensure a circumferentially correct orientation of the end cap 12 during the assembly of the housing 10 and the end cap 12 and to prevent rotation of the end cap 12 relative to the housing 10 in the snapped-on state. The housing 10 may have an elongation or broadening beyond the housing contour 10d in the proximal direction, which in the snapped-on state may be held as a whole by the end cap 12. The elongation of the housing 10 may be used for radial centering of the end cap 12 during the assembly of the housing 10 and the end cap 12 and may support the snap elements 10c of the housing 10. For instance, the elongation of the housing 10 may have a cross-section with the same cross-sectional shape as the housing 10 and may correspond to a cross-section of the end cap 12, at least on an inner cross-section, and thus the elongation of the housing 10 may also facilitate prevention of rotation of the housing 10 relative to the cap 12.

As illustrated in FIGS. 7a and 7b, the housing 10, the elongation of the housing 10 and the end cap 12 may have a square cross-section with slightly convexly rounded sides and a discrete rotational symmetry limited to four 90° positions. In contrast, the housing and cap contours 10d, 12b may have only a 180° symmetry, since, in each case, the contour sections on two opposite sides may be identical. Accordingly, in a preassembly position in which the end cap 12 is rotated by 90° with respect to the rotation position for the final assembly (FIG. 7b), the projections of the housing contour 10d may not engage in the recesses of the cap contour 12b, so that the end cap 12 can only be shifted up to an intermediate position onto the elongation of the housing 10. In this preassembly position, a holding cam 12c may snap on the inner side of the end cap 12 into a recess 10e of the housing elongation of the housing 10, so that the end cap 12 may be held sufficiently firmly at least for transport purposes, but can also be easily released from this connection for the final assembly.

The centering, orientation and snap elements provided herein may also be attached on the respective other component. For example, a concentric distal elongation of the end cap 12 may be provided instead of an elongation of the housing 10, and/or the snap elements 10c of the housing 10 may instead be attached to or formed by the end cap 12 and engage in a corresponding opening of the housing 10. As an alternative to a continuous elongation provided along the housing periphery, axially oriented arms may alternatively be provided, which may protrude from the proximal end of the housing 10 or from the distal end of the end cap 12, may support the snap elements, e.g., snap elements 10c, and may ensure a centering and/or alignment of the end cap 12 during the assembly. Instead of the releasable holding cams 12c, other connection means such as engagement elements or bayonet and threaded connections may also be provided for the preassembly, and, instead of the snap elements 10c, non-releasable connectors may also be used.

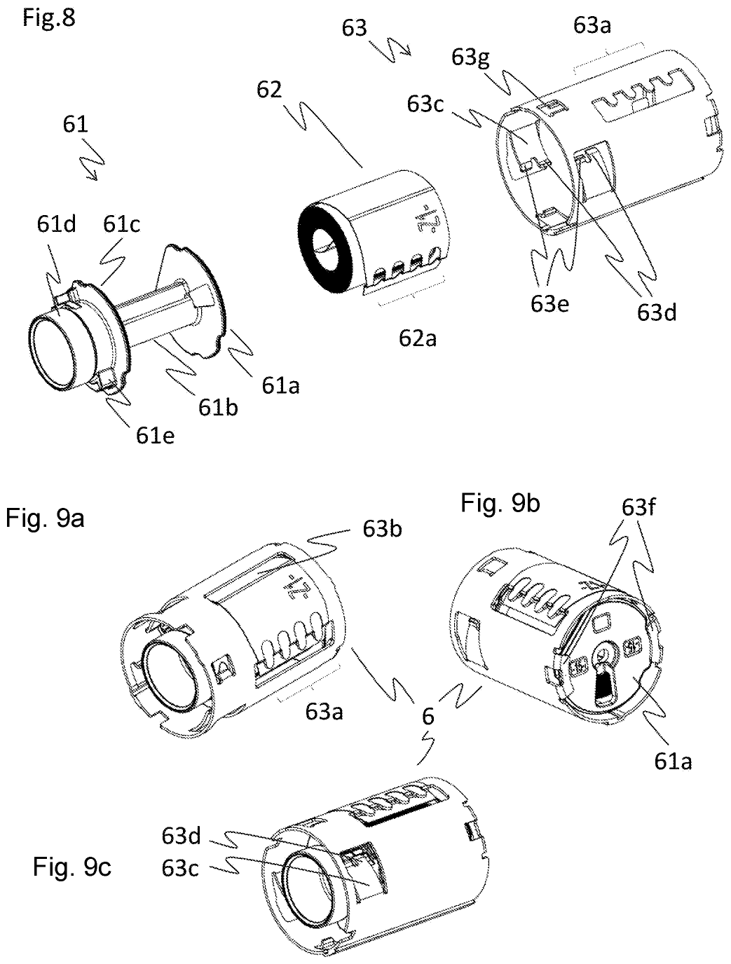

FIG. 8 shows an exploded representation of the spring pack 6 of the autoinjector of FIG. 1. The spring pack 6 may include a spring shaft 61, a spiral spring 62, and a spring sleeve 63. The spring shaft 61 may be configured as one-piece and may include a proximal end flange 61a, a spring axis 61b, a distal end flange 61c, and a sleeve-shaped distal extension 61d as a guide and for positioning with respect to a mechanism holder. On the distal end flange 61c, a stop 61e with a radially oriented stop surface may be provided. The spring shaft 61 may include a borehole in the direction of the spring axis, in which, during the assembly of the spring pack 6 on a drive unit, a proximal end of a threaded rod may be received for instance in a rotationally fixed manner. The spiral spring 62 may in the form of a coil from a wound spring strip made of spring steel and may be anchored with an inner end in the spring axis 61b in a rotationally fixed manner, and may be wound in the untensioned state in twelve windings around the spring axis, and may be connected at an outer end to the spring sleeve 63. The distance between the two end flanges 61a, 61c may correspond to the length of the spring axis 61b and the width of the spring strip. The inner end of the winding may include a recesses or holding tabs in the spring strip, and may be used for hooking or introducing into corresponding counter-elements of the spring axis 61b, such that at least torsion forces or a torque between the spring shaft 61 and the spiral spring 62 can be transmitted. The spring sleeve 63 may be configured as a sleeve-shaped cylinder to for instance define a sleeve jacket and may be made of sheet metal. The diameter and height of the spring sleeve 63 may be configured to receive the cylinder shaft 61 and spring sleeve 62. The spring sleeve 63 may include two mutually opposite locking catches 63c, each with a flexible arm and an inward pointing tooth 63d on the free end of the arm. In addition to the tooth 63d, and as part of the same flexible arm of the locking catch 63c, a control element 63e in the form of an outwardly angled tab may be provided. The spring sleeve may also include two mutually opposite and inward pointing stop elements 63g.

The spiral spring 62, e.g., a spring strip configured as a flat metal strip, may be anchored in the spring axis 61b and then be wound around the cylinder shaft 61 between the two end flanges 61a, 61c which flanges 61a, 61c may be permanently and firmly, e.g., non-detachably, connected to the spring axis 61b. Subsequently, the spring shaft 61 and the spiral spring 62 may be introduced in the proximal direction into the spring sleeve 63, and the spiral spring 62 may be connected to the spring sleeve 63. For this purpose, the outer end of the spiral spring 62 may include four openings, holes or eyelets 62a, which may lie next to one another along the spring axis in the spring strip, and the spring sleeve 63 may include four prongs or teeth 63a arranged next to one another in the form of a comb in the direction of the spring axis. The four prongs 62a of the spiral spring 62 may point in the spring tensioning direction and may be received by a complementary recess in the spring sleeve 63. The openings 62a of the spiral spring 62 may be pulled under slight tensioning of the spiral spring 62 via the prongs 63a of the spring sleeve 63 and may be hooked in or hooked on the prongs 63a. Due to the engagement of the toothing, an axial positioning of the spiral spring 62 with the spring sleeve 63 occurs.

FIG. 9a shows a first perspective representation of the spring pack 6 of FIG. 8. The outer end of the spiral spring 62 may be hooked by means of the four openings 62a in the spring strip into the four prongs 63a of the spring sleeve 63. For this purpose, the spring strip of the spiral spring 62 may first lead outward through a slot 63b of the spring sleeve 63, which may be different from the recess, so that the web, which connects the prongs 63a between the slot 63b and the recess to one another, is covered in FIG. 9a by the spring strip of the spiral spring 62. The spring strip, in the region of the openings 62a of the spiral spring 62, may include a stair step leading inward in the direction of the spring tensioning or winding, with a radial step height that may correspond to the thickness of the spring strip and the sleeve material, and the sleeve strip on both sides of the step may be oriented substantially tangential to the sleeve jacket. The stair step may include two bending edges in the spring strip; in-between a step extension of the spring strip may not be angled by more than 90° with respect to the spring tensioning direction. This stair offset may be arranged between the prongs on the web.

FIG. 9b shows a second perspective representation of the spring pack 6 of FIG. 8 illustrating another view. The proximal end flange 61a of the spring shaft 61 may be clearly seen. The proximal end flange 61a and thus also the spring shaft 61 may be held against proximal movements by a radially inward facing holding structure 63f of the spring sleeve 63. The proximal end flange 61a may include a radially extending slot or an inspection window for the optical control or inspection of the spiral spring.

Alternatively to the prongs 63a formed in the sleeve material of the spring sleeve 63, prongs or hooks protruding radially from the sleeve material may be attached or molded on the spring sleeve 63, e.g., on a sleeve jacket. If the prongs are applied to the inner side of the spring sleeve 63, recesses or slots in the sleeve material may need to be provided. The spring strip of the spiral spring 62 can also be led outward through the recesses, for instance by means of an outward leading stair step in the spring tensioning direction, so that the separate slot, e.g., slot 63b, in the spring sleeve 63 may be avoided. Likewise, more or fewer than four openings in the spiral spring 63 and prongs in the spring sleeve 63 may also be provided, and/or other forms of spiral spring 62 openings other than circular forms, such as openings formed with an edge parallel to the spring axis, together with corresponding hooks in the sleeve material of the spring sleeve 63, which may be configured to be wider. The number of openings in the spiral spring 62 may exceed the number of hooks in the spring sleeve 63. The openings, the recess and the web may be produced in a simple manner for instance punching or laser cutting from the spring strip of the spiral spring 62 and the sheet metal of the sleeve material of the spring sleeve 63.

FIG. 9c shows a third perspective representation of the spring pack 6 of FIG. 8 in a position rotated by 90° around the spring axis of the spring pack 6 with respect to the FIG. 9a. The spiral spring 62 may be tensioned counterclockwise (distal viewing direction) relative to the spring shaft 61 by rotation of the spring sleeve 63, and, in the end state, the spiral spring 62 may form, for example, three times more windings than in the untensioned state. The resulting pretensioning may correspond to a torque of 1 to 100 Nmm (Newton millimeter), such as a torque of 30 to 80 Nmm, or a torque of 60 to 70 Nmm. By appropriate measures, the potential energy generated by the tensioning process may be stored in the spring pack 6, and the spring pack 6 may be transported or stored as bulk product with the spiral spring 62 tensioned. For this purpose, the distal end flange 61c and the distal end of the spring sleeve 63 may include securing elements for securing a charged spiral spring 62 and/or may include coupling elements for the detachable coupling of the spiral spring 62 from the distal end flange 61c and the spring sleeve 63. The securing elements may include the stop 61e of the spring shaft 61 with a radially oriented stop surface on the distal end flange 61c, into which the inward facing tooth 63d on the free end of the flexible arm of the locking catch 63c of the spring sleeve 63 may engage. The locking direction of the engagement of the stop 61e of the spring shaft 61 and the tooth 63d of the spring sleeve 63 may be selected such that a relaxing of or tensioning loss in the spiral spring 62 may be prevented, and the spring sleeve 63 may be secured relative to the spring shaft 61 against rotation or turning by a torque of the spiral spring. In this position, the arm of the locking catch 63c may also limit a movement of the distal end flange 61c in the distal direction, analogously to the proximally limiting holding structure 63f of the spring sleeve 63. A movement of the control element 63e into a radial release position may result in a movement of the tooth 63d in the same direction; the rotation securing may thus be released due to a radial release movement of the control element 63e.

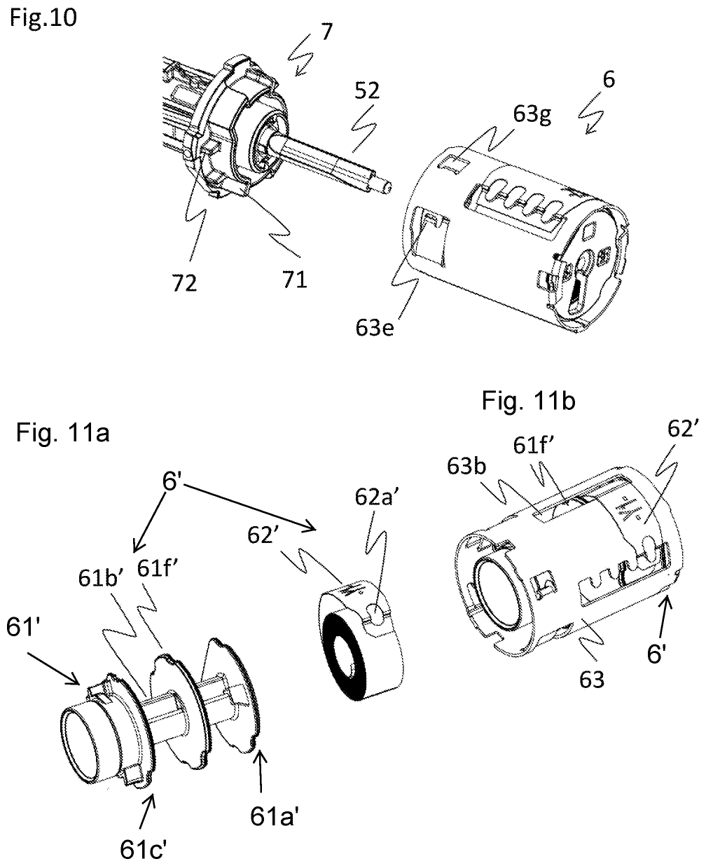

FIG. 10 shows a perspective representation of the spring pack 6 and a preassembled drive unit 7 of the injection device in a separated state. During the assembly of the spring pack 6 on the drive unit 7, the threaded rod 52 of the drive unit 7 may be introduced into the bore of the spring shaft 61, and a release element 71 of the drive unit 7 may be positioned radially between the stop 61e (FIG. 8) and the spring sleeve 63 and may be positioned slightly offset around the spring axis 61b toward the control element 63e. In a subsequent rotation of the spring pack 6 relative to the drive unit 7, the release element 71 may engage in the control element 63e of the spring sleeve 63 and may move the control element 63e and thus also the tooth 63d radially in the release direction. As a result, the spring sleeve 63 may be released for rotation with respect to the spring shaft 61 received in a rotationally fixed manner by the drive unit 7; and by a slight relaxation of the spiral spring 62, the spring sleeve 63 may be rotated until an inward facing stopping element 63g (FIG. 8) of the spring sleeve 63 abuts against a counter-abutment 72 of the drive unit 7 and a frictional and torque-locked connection between the spring pack 6 and the drive unit 7 may be established. Thereafter, the torque stored in the spring pack 6 may be coupled to the drive unit 7 and may be secured by locking elements in the drive unit 7 until the release of a product discharge.

In some implementations, the spring sleeve 63 may be formed from a metal sheet on which the locking catch 63c, which may be configured as a flexible arm, together with the tooth 63d and the control element 63e, may be formed by mechanical punching or laser cutting. On the free end of the arm of the locking catch 63c, the tooth 63d may be formed as a tab and may be bent inward and the control element 63e may be formed as a tab and may be bent outward. The stopping element 63g of the spring sleeve may also be cut out of the sleeve jacket of the spring sleeve 63 and may be bent inward. In the embodiments shown, two locking catches 63c and two stopping elements 63g with corresponding stops may be distributed over the periphery of the spring pack 6 and offset with respect to one another by 180° in each case. Here, it is also possible to provide only one locking catch 63c and/or stopping element 63g, or more than two locking catches 63c and/or stopping elements 63g.

After the spiral spring has been fully tensioned, the tooth 63d may be brought in engagement with the stop 61e, for example, by a plastic deformation of the arm of the locking catch 63c. Alternatively, an elastic deformation of the arm of the locking catch 63c is also conceivable, where the locking catch 63c may be blocked by the control element 63e by engagement with the stop 61e. The control element 63e may include least one engagement surface for the release element 71 for instance in the form of a threaded surface, a bevel or a wedge. The securing-releasing deflection of the control element 63e may occur in the radial direction as shown, or in the axial or tangential direction. The securing elements for rotational securing may also include a flexible locking catch on the spring shaft 61 and an abutment on the spring sleeve 63. The stop 61e or the locking catch 63c may also be positioned directly on the spring shaft 61, on an additional flange connected in a rotationally fixed manner to the flange, or on a spoke connected in a rotationally fixed manner to the shaft, at any desired radial distance from the spring shaft 61. The complementary locking catch or abutment may be brought to an appropriate distance from the spring axis 61b on a torque transmission means connected in a rotationally fixed manner to the spring sleeve 63. Likewise, the stopping element 63g of the spring sleeve 63 and the complementary counter-abutment 72 of the drive unit 7 may also be arranged at a small distance from the spring axis 61b. During the twisting of the spring pack 6, the release element 71 may also deflect or shift the control element 63e away axially instead of radially. The release movement of the control element 63e may also occur during the axial movement of the spring pack 6 and the drive unit 7, so that the subsequent twisting may be omitted. At least the proximal end flange 61a, as a radial boundary surface, may also be firmly connected to the spring sleeve 63 instead of the spring shaft 61.

FIG. 11a shows an exploded perspective representation of components of a second spring pack 6′ including a second spiral spring 62′ made of a spring strip with a width which is smaller with respect to the spiral spring 62 of the spring pack 6 of FIG. 8, and a spring shaft 61′. An intermediate flange 61f may be attached between the end flanges 61a′, 61c′ on the spring axis 61b′, and together with the proximal end flange 61a′ may define the spring volume. The spring sleeve 63 may be identical to that of the spring pack 6 of FIG. 8. The spring strip of the spiral spring 62′ may include only one opening 62a′ instead of four openings; and as a result, as shown in the assembled spring pack 6′ of FIG. 11b, the distal half of the hook-connecting web in the sleeve material of the spring sleeve 63 may include three unused prongs 63a and the slot 63b in the sleeve material both of which are clearly visible.

Instead of the spiral, clock or drive spring, the spring packs 6, 6′ may also include an energy store in the form of other tension, torsion or rotation springs, for example, a coil or helix spring wound from a spring steel, in which the potential energy necessary for the product discharge may be charged by applying a torque between the two spring ends. As an alternative to providing an autoinjector with a non-adjustable dose, the spring packs 6, 6′ may be provided in an automatic injection device configured with dose setting functions, also referred to as autopens.

The patent application publication WO2016/205963 described herein also discloses a spring pack that includes a spring shaft with a distal end flange, a spiral spring, a spring sleeve and a spring sleeve cover, which, depending on a spiral spring width, can be detachably fastened in different axial positions on the spring shaft. The spring shaft has an axially formed holding rib, in which the inner end of the spiral spring, configured as a holding tab, can be anchored in a rotationally fixed manner, and the spring sleeve has an axially oriented holding edge, on which the outer end of the spiral spring, which is configured as a holding tab, can be anchored in a rotationally fixed manner. The holding tab includes a holding tongue, which, in the case of a bending edge, is angled in the spring strip by more than 90° with respect to the spring tensioning direction and which faces in the direction toward the inner end of the spiral spring. For the anchoring, the holding tab is tensioned via the holding edge and, with relaxation of the spring tensioning, guided so that it hooks to the holding edge. The bending edge in the spring strip is stressed even more strongly by the twisting of the spring sleeve, which occurs after the anchoring relative to the spring axis for loading potential energy into the spiral spring.

A spring pack for an injection device according to prior approaches includes the following elements:

a spring shaft with a spring axis, a torsion or rotation spring, in particular a spiral or drive spring wound from a spring strip, and a spring sleeve, where the torsion spring is connected in a rotationally fixed manner by an inner end with the spring shaft and by an outer end with the spring sleeve in each case with respect to the spring axis;

a first and a second securing element, which are firmly arranged on the spring sleeve and on the spring shaft and, in engagement, secure the spring sleeve in a rotationally fixed manner relative to the spring shaft with respect to the spring axis, where the engagement can be released by a release movement of the first securing element;

a control element which, during the assembly of the spring pack, can be moved onto a drive unit of the injection device by a release element of the drive unit, so that the first securing element performs a release movement and a torque stored in the spring pack is coupled to the drive unit.

According to implementations of the present disclosure, the spring packs 6, 6′ may be further configured such that:

a) the outer end of the spiral spring 62, 62′ may form a loop or have an opening, which, with tensioning of the spiral spring 62, 62′ in a spring tensioning direction, e.g., by gripping, tensioning or guiding of the outer end of the spiral spring strip, may be hooked to a prong or a hook of the spring sleeve 63, e.g., prongs 63a;

b) the prong of the spring sleeve 63 may point in the spring tensioning direction and may be formed by a recess in a sleeve material of the spring sleeve 63;

c) the spiral spring strip of the spiral spring 62, 62′ may have a step in the region of the opening, and may have a step height which may be at least approximately equal to a thickness of the spiral spring strip of the spiral spring 62, 62′ and of the sleeve jacket of the spring sleeve 63;

d) the first securing element, e.g., the locking catch 63c of the locking sleeve 63, may perform a release movement in the direction of the spring axis; and/or

e) the spring strip of the spiral spring 62, 62′ may be arranged between two end flanges 61a, 61a′, 61c, 61c′ and may be connected in a nondetachable and/or axially fixed manner to the spring shaft 61, 61′.

With the disclosed spring packs 6, 6′, a group of modular spring pack assemblies may be provided, which may include a first and a second spring pack, such as spring packs 6, 6′, in which the first spring pack, e.g., spring pack 6, may include a first spring shaft with two firmly attached flanges, a first spiral spring which may be wound between the two flanges around the spring shaft and the width of which may correspond to the distance of the flange, and a spring sleeve which may surround the spiral spring; and in which the second spring pack, e.g., spring pack 6′, may include a second spring shaft with two firmly attached flanges, a second spiral spring, which may be wound between the two flanges around the spring shaft and the width of which may correspond to the distance of the flange, and a spring sleeve, which may surround the second spiral spring. The first and the second spring shafts, such as spring shafts 61, 61′ as well as the widths of the first and the second spiral springs, such as spiral springs 62, 62′ may be different, but the spring sleeve, such as spring sleeve 63, and thus the shape and the coupling elements with the drive unit 7 may be identical across the two spring packs. Springs having a different spring strip width may thus also be assembled in an administration device, and may avoid the need to otherwise adjust the device. Thus, identically constructed administration devices may be provided with the differing spring packs providing for an adjustment of the force to be exerted during the administration of a certain drug.

LIST OF REFERENCE NUMERALS

| | |
|---|---|
| 10 | Housing |
| 10a | Recesses or inspection window |
| 10b | Holding portion |
| 10c | Snap element |
| 10d | Housing contour |
| 10e | Recess |
| 11 | Syringe holder |
| 11a | Support element |
| 11b | Finger |
| 11c | Holder sleeve |
| 11d, e, f | Snap element |
| 11g | Assembly supporting element |
| 12 | End cap |
| 12a | Opening |
| 12b | Cap contour |
| 12c | Holding cam |
| 13 | Mechanism holder |
| 13a | Holding spring portion |
| 14 | Adapter |
| 14a | Support element |
| 14b | Finger |
| 14c | Adapter body |
| 15 | Adapter holder |
| 15a | Holder sleeve |
| 15b | Holding portion |
| 15c, d, e | Snap elements/securing elements |
| 2 | Ready-to-use or pre-filled syringe |
| 21 | Syringe body |
| 22 | Syringe shoulder |
| 23 | Piston or stopper |
| 24 | Product receiving portion |
| 25 | Injection needle |
| 26 | Finger flange |
| 27 | Needle protection cap |
| 72 | Counter-abutment |
| 30 | Pull cap |
| 31 | Protection cap remover |
| 40 | Needle protection sleeve |
| 40a | Sleeve arm |
| 40b, c | Recess |
| 41 | Needle protection sleeve spring |
| 42 | Switching sleeve |
| 43 | Locking sleeve |
| 50 | Plunger rod |
| 50a | Recess |
| 51 | Holding element |
| 51a | Holding arm |
| 51b, c | Engagement element |
| 51d | Projection |
| 52 | Threaded rod |
| 6, 6′ | Spring pack |
| 61, 61′ | Spring shaft |
| 61a, a′, c, c′ | End flange |
| 61b, 61b′ | Spring axis |
| 61d | Extension |
| 61e | Stop |
| 61f′ | Intermediate flange |
| 62, 62′ | Spiral spring |
| 62a, 62a′ | Openings |
| 63 | Spring sleeve |
| 63a | Prong |
| 63b | Slot |
| 63c | Locking catch |
| 63d | Tooth |
| 63e | Control element |
| 63f | Holding structure |
| 63g | Stopping element |
| 7 | Drive unit |
| 71 | Release element |
| 72 | Counter-abutment |

What is claimed is:

1. A modular syringe holder, comprising:
an adapter holder configured to be received in a housing of an injection device; and
an adapter comprising an adapter body and a support element flexibly coupled to the adapter body,
wherein the support element of the adapter is configured to receive, along a longitudinal axis of the syringe holder, a syringe shoulder of a pre-filled syringe, and
wherein the adapter holder is configured to receive the adapter such that the support element is blocked by a holding portion of the adapter holder from deflecting in a radial direction.

2. The modular syringe holder according to claim 1, wherein the support element is flexibly coupled to the adapter body such that the support element is configured to be deflected in the radial direction by a needle protection cap of the pre-filled syringe prior to the adapter being received in the adapter holder.

3. The modular syringe holder according to claim 1, wherein the adapter further comprises a flexible finger, and wherein the support element is coupled to a distal end of the finger and protrudes from the finger in a direction transverse to the longitudinal axis.

4. The modular syringe holder according to claim 1, wherein the adapter and the adapter holder are configured such that the support element directly contacts the housing of the injection device in an axial direction.

5. The modular syringe holder according to claim 1, wherein the adapter holder is secured by a plurality of securing elements in an axially and rotationally fixed manner in the housing of the injection device, and wherein the adapter and the adapter holder are configured to enable a rotation of the pre-filled syringe about the longitudinal axis.

6. The modular syringe holder according to claim 5, wherein the adapter and the adapter holder are configured such that the support element directly contacts the housing of the injection device in an axial direction.

7. The modular syringe holder according to claim 6, wherein the support element is flexibly coupled to the adapter body such that the support element is configured to be deflected in the radial direction by a needle protection cap of the pre-filled syringe prior to the adapter being received in the adapter holder.

8. The modular syringe holder according to claim 1, wherein the adapter further comprises two support elements and two flexible fingers, and wherein each of the flexible fingers comprises one of the support elements and such support element is configured for receiving axial forces of the syringe shoulder.

9. A method for assembling a pre-filled syringe in an injection device, the injection device comprising a housing defining a longitudinal axis, the method comprising:
inserting a syringe holder into the housing of the injection device in a first axial position relative to the housing, wherein in the first axial position a support element of the syringe holder is flexibly coupled to a holder sleeve of the syringe holder such that the support element is radially deflectable;
blocking the syringe holder against a movement in a distal direction;
introducing a pre-filled syringe into the syringe holder in the distal direction, wherein the pre-filled syringe causes the support element to undergo a radial deflection by a needle protection cap of the pre-filled syringe;
releasing the blocked movement of the syringe holder in the distal direction; and
moving the syringe holder into a second axial position such that the support element is blocked by a holding portion of the housing against the radial deflection.

10. The method of claim 9, wherein the syringe holder is blocked against a movement in the distal direction by an assembly tool engaging on an assembly supporting element of the syringe holder.

11. The method of claim 10, further comprising introducing the assembly tool perpendicular to the longitudinal axis through an inspection window of the housing.

12. The method of claim 9, wherein the pre-filled syringe comprises a product receiving portion and an injection needle nondetachably fastened thereon, wherein the product receiving portion comprises a nominal filling volume of less than 2.25 mL, and wherein the pre-filled syringe is axially nonshiftably held in the housing of the injection device.

13. An injection device comprising a modular syringe holder with a pre-filled syringe inserted therein, wherein the injection device is configured as an autoinjector, the modular syringe holder comprising:
an adapter holder received in the housing of an injection device; and
an adapter comprising an adapter body and a support element flexibly coupled to the adapter body, wherein the support element is flexibly coupled to the adapter body such that the support element is configured to be deflected in the radial direction by a needle protection cap of the pre-filled syringe prior to the adapter being received in the adapter holder, wherein the support element of the adapter is configured to receive, along a longitudinal axis of the syringe holder, a syringe shoulder of the pre-filled syringe,
wherein the adapter holder is configured to receive the adapter with the pre-filled syringe inserted therein such that a holding portion of the adapter holder blocks the support element from deflecting in a radial direction,
wherein the pre-filled syringe comprises a product receiving portion and an injection needle nondetachably fastened thereto, wherein the product receiving portion comprises a nominal filling volume of less than 2.25 mL, and
wherein the pre-filled syringe is axially nonshiftably held in the housing of the injection device by the modular syringe holder.

14. The injection device according to claim 13, wherein the adapter and the adapter holder are configured such that the support element directly contacts the housing of the injection device in an axial direction.

15. The injection device of claim 13, wherein the adapter and the adapter holder are configured to enable a rotation of the pre-filled syringe about the longitudinal axis.

16. The injection device of claim 13, wherein the injection device comprises a drive with a spiral spring, wherein in a delivery state, the spiral spring stores at least an amount of energy for a complete discharge of a product in the product receiving portion.

17. The injection device of claim 13, wherein the injection device comprises a drive, the drive comprising a spring pack with a spring shaft, a spiral spring, and a spring sleeve,
wherein the spiral spring is rotationally fixed with respect to the longitudinal axis and connected by an inner end to the spring shaft and connected by an outer end to the spring sleeve,
wherein a first securing element is arranged on the spring sleeve and a second securing element is arranged on the spring shaft, wherein when the first securing element is in an engagement with the second securing element, the spring sleeve is secured in a rotationally fixed manner relative to the spring shaft with respect to the longitudinal axis, wherein the engagement can be released by a release movement of the first securing element;
wherein during the assembly of the spring pack onto a drive unit of the injection device, a control element of the spring pack can be moved by a release element of the drive unit such that the first securing element performs the release movement and a torque stored in the spring pack is coupled to the drive unit.

18. The injection device of claim 17, wherein the outer end of the spiral spring comprises an opening which, with tensioning of the spiral spring, can be hooked in a prong of the spring sleeve.

19. The injection device of claim 17, wherein the drive is configured to receive one of a plurality of different spring packs, wherein a first spring pack of the plurality of different spring packs is configured with a first torque stored in the first spring pack, and wherein a second spring pack of the plurality of different spring packs is configured with a second torque stored in the second spring pack that differs from the first torque.
